(19) United States
(12) Patent Application Publication
(10) Pub. No.: US 2021/0346658 A1
(43) Pub. Date: Nov. 11, 2021

(54) ACTIVE-SUBSTANCE COATING FOR BALLOONS OF BALLOON CATHETERS

(71) Applicant: Alexander RUEBBEN, Monaco (MC)

(72) Inventor: Alexander RUEBBEN, Monaco (MC)

(21) Appl. No.: 17/277,316

(22) PCT Filed: Sep. 11, 2019
(86) PCT No.: PCT/EP2019/074158

§ 371 Date: Mar. 18, 2021

(30) Foreign Application Priority Data
Sep. 19, 2018 (DE) 10 2018 123 050.8

(51) Int. Cl. A61M 25/10; A61L 29/08; A61L 29/16; A61L 29/04

(52) U.S. Cl. CPC A61M 25/1029; A61M 25/104; A61L 29/085; A61L 2420/02; A61L 29/043; A61M 2025/1031; A61L 29/16

(57) ABSTRACT

The invention relates to a method for coating the surface of the balloon of a balloon catheter with an active substance, with the balloon being made of an elastic material and being expandable by pressurization with a fluid, with the balloon being designed for expansion at a target site, wherein the coating of the surface of the balloon being applied at a pressure which is lower than the pressure used to expand the balloon at the target location. In this way, due to the generation of shear forces a particularly effective delivery of the active substance from the balloon to the inner wall of the blood vessel or the surrounding tissue is achieved. Furthermore, the invention relates to a relevant balloon as well as a balloon catheter.

The invention relates to a method of coating the surface of the balloon of a balloon catheter with an active substance or agent, wherein the balloon is made of an elastic material and is expandable by pressurization with a fluid, with the balloon being intended for dilatation at a target location.

In medicine so-called “minimally invasive procedures” play an ever-increasing role. Percutaneous transluminal angioplasty (PTA) by means of balloon dilatation is frequently employed for the treatment of vasoconstrictions such as arteriosclerosis. For this purpose, a balloon catheter provided in its distal area with a balloon inflatable by the infeed of a fluid is brought to the stenotic site (vasoconstriction) with the aid of a guide catheter. At the stenotic site the balloon is inflated causing deposits/plaque inhibiting the flow of blood to be pressed against or into the vessel wall so that unhindered perfusion is restored. When the treatment has been completed and the balloon is subsequently folded, the balloon catheter is withdrawn from the vascular system and removed.

In some cases, an angioplasty successfully carried out initially may be followed by re-narrowing in the treated vessel segment. As a rule, such restenosis is due to cell proliferation in the respective vessel segment resulting in cells of the blood vessel growing into the vessel lumen and in turn causing an obstruction of the blood flow. To prevent this, balloon catheters coated with medications are increasingly being put to use. In most cases, suitable drugs have an inhibitory effect on proliferation, particularly on the smooth muscle cells (SMCs), and are thus intended to prevent restenosis caused by an excessive growth of these cells. The medication is applied to the outside of the balloon and is transferred during balloon dilatation from the balloon to or into the inner vessel wall.

Typically, the balloon of the balloon catheter is coated by applying an active agent dissolved in a solvent onto the surface of the balloon, with the solvent evaporating after application of the solution. The active agent is present on the surface in the form of a layer and can be applied while balloon dilatation takes place.

Transferring of the active substance from the surface of the balloon to the inner wall of the vessel has proven to be problematic. Here, it must be borne in mind that the expansion of the balloon must be narrowly limited in time, because occlusion of the blood vessel for too long a period would cause ischemia and under-supply of the tissue or organs, and may even result in infarction. Accordingly, the transfer of the active substance must take place within a comparatively short period of time. In the coronary region, the allowable maximum period for balloon expansion is 30 to 60 s. It is to be noted however, that known medication-coated balloons often require a longer period of time for adequate drug delivery. This leads either to the ischemic problems referred to above or to an insufficient drug delivery due to a necessary shortening of the balloon expansion time.

Moreover, it must be ensured that the active substance only separates from the balloon surface at the target site, and this is especially important since the active agents used are often toxic substances such as paclitaxel, for which a release away from the target site is undesirable. Likewise important is that safety must as well be ensured for the medical staff performing the treatment. Hence, there is a conflict of goals: on the one hand, the active substance or medication must be released at the target site and transferred to the inner wall of the blood vessel as quickly as possible, but on the other hand, the active substance should adhere as firmly as possible to the balloon during preparation and advancement of the balloon catheter. However, the release of a firmly adhering active substance is usually rather slow.

It is therefore the objective of the present invention to provide a balloon which, on the one hand, securely retains the active substance coating, but, on the other hand, is capable of rapidly releasing the active agent when balloon expansion takes place at the target site.

As proposed by the invention, this objective is achieved by a method for coating the surface of the balloon of a balloon catheter with an active substance, wherein the balloon being made of an elastic material and being expandable by pressurization with a fluid, with the balloon being designed for expansion at a target site, and with the coating of the surface of the balloon being applied at a pressure which is lower than the pressure used to expand the balloon at the target location.

With regard to balloons for balloon catheters, a fundamental distinction must be made between non-compliant, semi-compliant and compliant balloons. The difference between the balloons is the different increase in diameter when the balloon is filled with a fluid under a certain pressure. Compliance in this context is defined as follows:

Compliance (in %)=(d(high pressure)−d(low pressure))/d(low pressure)×100%, where d is the diameter of the balloon.

The high and low pressure levels can be considered to be the end points of the so-called working range, with this working range extending between the rated pressure at which the balloon reaches its nominal diameter and the maximum pressure to which the balloon can be brought without suffering damage. Depending on the percentage of diameter increase, the following distinctions are made:

non-compliant balloons, diameter increase (compliance): 0 to 7%,

semi-compliant balloons, diameter increase (compliance): 5 to 10% and

compliant balloons, diameter increase (compliance): 10 to 500%.

Regarding the distinction between non-compliant, semi-compliant and compliant different definitions may sometimes be found in literature, but the basic rule is that the diameter of a compliant balloon increases significantly with pressure, while the diameter of a non-compliant balloon remains virtually constant even at high pressure. Non-compliant balloons are made of a material that is substantially inelastic. With respect to their increase in diameter, the characteristics of semi-compliant balloons range between those of non-compliant and compliant balloons.

All these balloons can be usefully employed to cover numerous applications needs. In addition, all balloons have certain advantages and disadvantages. Compliant balloons are more flexible and can therefore be easier inserted and are suitably used in applications where adaptation to the shape of a vessel is desired, also to treat far advanced vasoconstriction, and for pre-dilatation prior to stent placement. On the other hand, a non-compliant balloon is indicated in the event the balloon is to be brought to a specific diameter over its entire length, for example, to push very firm, calcified vessel wall deposits outwards or to press an already placed stent firmly and evenly towards the vessel wall (post dilatation). When pressed against vessel wall deposits, a non-compliant balloon exhibits an essentially cylindrical structure, whereas a semi-compliant or compliant balloon expands more significantly on the proximal and distal side of the deposit than in the area of the deposit itself when pressed against deposits that extend only over a short section. In this context, this characteristic is also known as dog bone effect.

Since the balloon surface A, at least in the cylindrical region with the balloon being expanded, is determined by the equation

A=π×d×L

where d refers to diameter and L to length, the surface area increases at the same rate as the diameter, assuming a constant length of the balloon, meaning, the definitions of compliance stated hereinbefore apply in the same way to the balloon surface. Whenever the diameter of the balloon is mentioned in the framework of this invention, this shall always be understood to denote its outer diameter.

The invention relates to semi-compliant or compliant balloons, i.e. balloons made of a material that offers certain elasticity. As proposed by the present invention, such a balloon is coated with an active substance at low pressurization, whereas the pressure at which the balloon is dilatated at the target site is higher. As regards a balloon having a certain compliance, this leads to an increase in diameter and thus also in the balloon surface. Due to the fact that the pressure at the target site, where the active medication applied to the balloon surface is to be delivered to the inner vessel wall, is higher than the pressure at which the balloon was coated with the active substance, shear forces occur within the active substance layer. Therefore, the active substance is at least partially split off and thus detaches from the balloon. In this way, even strongly adhering coatings can be removed.

The balloon catheters proposed by the invention can be used in blood vessels, particularly in the field of angioplasty. In this case, the target site of the balloon is a blood vessel. However, using balloon catheters in other medical settings is also possible. One of the possible applications is in urology, where balloon catheters are inserted into the urinary bladder as bladder catheters, with the catheter being fixed in place by means of the balloon. The balloon in this case may be provided for instance with a coating that prevents bacterial colonization and incrustation, for example using heparin.

In pulmonology, balloon catheters can be used to dilatate or occlude a bronchus. Balloon catheters can also be employed in the field of gynecology. In the field of orthopedics, balloon catheters can be put to use for the treatment of vertebral fractures with a view to realigning the vertebrae by means of balloon expansion techniques (balloon kyphoplasty). In principle, the inventive balloon catheter can be employed in all fields of medicine in which coated balloon catheters are the preferred choice.

By preference, the invention proposes that balloons are used in which, starting from the rated pressure at which the balloon reaches its normal or nominal diameter, the diameter increase when doubling the pressure is at least 5%, preferably at least 10%, further preferably at least 20% and particularly preferred at least 30%.

The invention offers the additional advantage that the detachment of the coating does not occur until the dilatation of the balloon takes place, which means, only at the target site where the active substance is actually to be transferred, for example to the inner wall of the blood vessel. In contrast, when the balloon is in compressed state, no active substance release/detachment occurs; in other words, the release of active substance is precluded in areas of the vascular system where this is not intended. Likewise, there is no release or detachment of active substance outside the body that may be dangerous for persons coming into contact with the balloon catheter.

The expansion of the balloon is thus deliberately utilized to transfer active substance from the balloon, in particular, to the inner wall of a blood vessel. To accomplish a uniform coating of the balloon, the pressure which is exerted on the balloon to produce the coating should be high enough to cause the balloon to unfold completely or at least to a large extent, but should be lower than the pressure typically applied to the balloon at the target site in the blood vessel. For example, a balloon can be coated using a pressure of 3 bar, while the pressurization in the blood vessel amounts to 6 bar. The diameter of a compliant balloon can increase in this case, for example, from 4.5 mm to 6 mm, which translates in an increase in diameter and thus also in balloon surface area of 33%. This produces a strong shear force causing the active substance to spall off and be detached.

Advantageously, the pressure at which coating takes place is at least 20%, further preferably at least 30%, below the pressure that is exerted when the balloon is dilatated at the target site. More advantageously, the pressure at which coating takes place amounts to a maximum of 50% of the pressure that is applied to expand the balloon at the target site. The increase in balloon surface area between the coating process and the expansion process at the target location should amount to at least 10%, advantageously at least 20%, further advantageously at least 30%, further advantageously at least 40%, and further advantageously at least 50%.

In order to ensure the expansion capability underlying the invention, the balloon is at least partially made of an elastic material. As elastic material in this case a polyurethane, a polyolefin copolymer, a polyethylene, or a silicone can be used, for example. Other materials that can be employed are thermoplastic elastomers, in particular polyether block amides (PEBA). This is a thermoplastic elastomer obtained by the polycondensation of a carboxylic acid polyamide with a polyether with terminal OH groups. In particular, PEBA is sold by the company of Arkema under the tradename of PEBAX®. Polyamides such as nylon (polyhexamethylene adipamide) with some elasticity can also be used, at least for semi-compliant balloons.

Alternatively, other polyamides may also be used as elastic material for the balloon, for example those available from the company of EMS-GRIVORY under the tradename of Grilamid®. Especially preferred is the use of a polyamide 12 (PA 12, Grilamid® L), a polyamide obtained by the polycondensation of laurolactam. Moreover, further conducively usable polyamides are polyamide 10.10 (PA 10.10, Grilamid® 1S), a polyamide obtained by polycondensation of decandiamine and sebacic acid, polyamide 6.10 (PA 6.10, Grilamid® 2S), a polyamide obtained by polycondensation of hexamethylenediamine and sebacic acid, or polyamide 6.12 (PA 6.12, Grilamid® 2D), a polyamide obtained by polycondensation of hexamethylenediamine and dodecanedioic acid.

The active substance used is, in particular, a drug or medical substance, preferably a medicinal product that has a proliferation-inhibiting effect preventing a vasoconstrictive overgrowing of the vessel location previously expanded by the balloon. Similarly, it may be a hormone-like or regulatory agent capable of influencing organ-specific effects or regulatory functions on certain cells. The active substance or agent may in particular be selected from the following: Tretinoin, orphan receptor agonists, elafin derivatives, corticosteroids, steroid hormones, paclitaxel, rapamycin, tacrolimus, hydrophobic proteins as well as substances modifying cell proliferation. Mixtures of these active substances may also be used. Moreover, derivatives of the above cited active agents may also be of use, wherein said derivatives may in particular be salts, esters, and amides. As steroid hormones methylprednisolone, dexamethasone or estradiol may be used, for example. Particularly preferred is the use of paclitaxel, rapamycin or tacrolimus or corresponding derivatives.

Generally speaking, however, the term active substance or agent is to be understood broadly, i.e. it can basically refer to any coatings on the balloon of the balloon catheter that are intended to achieve a specific effect at the target site. When introduced into blood vessels, this effect may in particular be focused on inhibiting cell proliferation. In other medical fields, however, the desired effect may be different, for example in the field of urology involving bladder catheters, where the coating is intended to serve in particular to inhibit bacterial colonization, in which case heparin may be employed as active substance, for example.

Coating the surface of the balloon with the active substance is typically achieved by bringing the surface of the balloon in contact with a solution of the active substance, which can be done in particular by immersing the balloon in the solution. Usually, the immersion does not take more than 1 minute, typically ranges between 10 and 30 s. After immersion, the balloon should be drawn out of the solution at a rate of up to 10 mm/s. Even more favorable would be to withdraw the balloon at a speed of less than 5 mm/s, preferably at a speed ranging between 0.5 mm/s and 2 mm/s. Withdrawing the balloon slowly enables the surface to dry gradually and slowly.

Before the balloon coating process commences, it is expedient to clean the surface of the balloon. This can be done, for example, using an appropriate solvent, such as the solvent which is also used for the application of the active agent.

The solution may be saturated with respect to the active agent, but this is not a mandatory requirement. Solvents that can be employed are, for example, methylene chloride, chloroform, alcohol, in particular ethanol, methanol or isopropanol, acetone, diethyl ether, liquid hydrocarbons, such as, for example, pentane, hexane, heptane, cyclohexane or octane, toluol, tetrahydrofuran (THF) or ethyl acetate. Furthermore, solvent mixtures or blends may also be employed. Preferably, this means a solution of the active agent in methylene chloride.

As an alternative to coating the balloon by immersion other methods may also be adopted, for example spraying.

Using the inventive balloon catheter involves the catheter being introduced into the blood vessel system or another body lumen and then inflated and in this way pressed against the inner wall of the vessel/lumen. In the process, a high proportion of the coating is transferred to the inner wall. After releasing the pressure and removing the balloon catheter from the vasculature/lumen, the active substance contained within the coating gradually penetrates into the tissue.

The term balloon as it is used within the scope of the present invention shall be understood to define the element of a balloon catheter that can be expanded by feeding in a fluid, irrespective of the shape or material of said expandable element. The fluid may be of gaseous or liquid nature. Preferred is a gas, for example air. The pressure applied to the balloon to bring about its expansion in the blood vessel/lumen typically ranges between 5 and 15 bar. The dimensions of the balloon may vary greatly depending on the field of application; for example, the diameter in the expanded state may range from approx. 1 mm to approx. 50 mm, and the length may range between approx. 5 mm and approx. 300 mm. However, the dimensions may also deviate from this, for example, when using the balloon/balloon catheter for applications in urology or veterinary medicine.

Basically, balloon catheters are sufficiently known from prior art and comprise an elongated catheter extending from proximal to distal as well as a balloon which is arranged distally. With respect to its dimensions such a catheter is suitably designed for the insertion into a body lumen, especially into a (blood) vessel system. The relevant dimensions of such catheters may vary depending on whether the blood vessel, for example, is a coronary artery, an intracranial blood vessel or an artery in the lower leg. Moreover, the balloon catheter is provided with means for supplying a fluid to the balloon. This may be a supply lumen extending over the length of the balloon catheter.

Furthermore, the inventive balloon catheter may not only serve for the elimination of stenoses and local administration of active substances but additionally for the placement of a stent (endoprosthesis) in a body lumen. Stents are tube-like supporting structures implanted into a body lumen, for example a blood vessel, with a view to keeping it permanently open. Stents of this nature may be of self-expanding design or expanded with the help of a balloon. For this purpose, the stent is crimped onto the balloon and introduced into the body lumen with the aid of a balloon catheter. At the desired placement site the balloon is inflated by feeding in a fluid, which also causes the stent to expand and thus be anchored in the body lumen. Using the inventive balloon enables the relevant active substance to be applied at the same time to the wall of the body lumen. Finally, the balloon is deflated and removed from the body lumen whereas the stent remains in the lumen.

As per a preferred embodiment at least the part of the balloon surface coated with the active substance is wetted with a water and/or at least one alcohol containing liquid. When coating the surface of the balloon with an active substance, a varnish-like, transparent layer of the active substance is usually created on the surface, which serves as basis for a homogenous and reproducible active agent loading. This coating is attacked by the liquid containing water and/or at least one alcohol which results in the surface becoming more porous or partially brittle. The entire coating thus becomes more brittle and less transparent visually, that is it has a milkier appearance. The surface so produced has a chalk-like, possibly even a non-crystalline consistency, which enables a higher active substance release/removal in the case of friction than can be achieved with a coating produced just by wetting the surface of the balloon with a solution of the active agent. A relevant method is known in principle from publication WO 2013/178820 A1.

The water and/or at least one alcohol containing liquid is, in particular, an aqueous solution containing an alcohol and/or ketone. The concentration of the alcohol and/or ketone in the aqueous solution typically ranges between 10 and 70% (v/v), preferably between 30 and 65% (v/v), further preferred between 50 and 60% (v/v), and most preferably approx. 55% (v/v). Basically suited for use are alcohols and ketones that can be mixed with water, with a blend consisting of several alcohols and/or ketones may also be employed, in which case the above indicated preferred concentration details shall apply as a whole. Preferred is the use of ethanol, methanol, acetone and/or isopropanol, with ethanol being mostly preferred. Moreover, the aqueous solution may comprise an azeotropic solvent blend, in particular an alcohol/water mixture, with an ethanol/water mixture being preferred. Also conceivable would be to provide the water and/or at least one alcohol containing liquid with an additional amount of active agent to increase the amount of active substances present on the balloon.

According to another advantageous embodiment of the invention, at least the part of the surface of the balloon coated with the active substance is provided with a coat of polysaccharide before or after the active agent coating is applied. It is also possible to initially apply a coat of active substance, followed by a coat of a polysaccharide, and finally apply another coat of active substance. Surprisingly, the polysaccharide coating was found to act similar to an adhesive on the inner wall of the treated vessel, that is, the medication adheres significantly better to the vessel wall and is less easily entrained by the blood stream. Accordingly, the active substance may remain effective over a long period of time and, out of the polysaccharide coating, is capable of penetrating gradually into the tissue of the vessel. It has been demonstrated that significant concentrations of the active substance could still be detected after some weeks.

Polysaccharides form a hydrophilic coating that undergoes a certain swelling or softening process when being present in an aqueous environment such as blood. During balloon dilatation this results in the active substance to be properly transferred onto the inner wall of the vessel. The method proposed by the invention is particularly suitable for applying lipophilic coatings to the balloon. It has indeed been found that hydrophilic polysaccharides in particular are well suited to cause lipophilic active substances to be effectively transferred during balloon dilatation to the inner walls of the vessels to be treated where they will bring about a long-lasting concentration of active agents.

Also, when applying a coating of polysaccharide, it is preferably provided in a solution, preferably an alcoholic solution. Aside from one or several alcohols this solution may in particular also contain water. An aqueous-alcoholic solution offers advantages in that it allows the polysaccharide to be well dissolved but does not affect or remove an already previously applied active agent layer. Moreover, the organic constituents present in the solution ensure rapid drying to take place after wetting. The concentration of the alcohol or alcohols in the additional solution typically ranges between 10 and 70% (v/v), preferably between 30 and 65% (v/v), further preferred between 50 and 60% (v/v), and especially preferred is approx. 55% (v/v). Suitable alcohols are those that are capable of dissolving polysaccharide. As a rule, such alcohols can also be mixed with water. Preferred are ethanol, methanol, and isopropanol, with ethanol being especially preferred.

Expediently, the mean molar mass of the polysaccharide ranges between 10,000 and 100,000,000 Da. A mean molar mass of between 20,000 and 80,000 Da has proved to be especially suitable for the purpose. The content of polysaccharides of the further solution preferably amounts to between 1 and 15% (w/w), further preferred between 2 and 10% (w/w), and especially preferred between 3 and 8% (w/w).

Preferably, the polysaccharide is a branched polysaccharide. Also suitable are mixtures composed of several polysaccharides and modified polysaccharides. Preferred are dextrans, in particular natural dextrans. Dextrans are high-molecular, branched polymers composed of glucose units. They are produced, inter alia, by bacteria of genus Leuconostoc and used as blood plasma substitutes or as carriers in the field of chromatography.

The dextran used may in particular be a natural dextran, and especially preferred is dextran 40 having a mean molar mass of approx. 40,000 Da. However, aside from dextrans other polysaccharides may basically be employed as well. An example of a modified polysaccharide that can be used is hydroxyethyl starch (HES).

In principle, either the entire balloon surface or only part of said balloon surface, for example only that area of the surface that will have contact with the tissue when the balloon has been inflated, can be coated by the inventive method. The balloon may, in particular, have a cylinder-shaped area and at least one tapering/conical area. In this case, for example, only the cylinder-shaped portion of the balloon may be coated with an active substance in accordance with the invention or the cylinder-shaped portion and a conical area.

Also, the wettings or coatings of the surface of the balloon with a liquid containing water and/or at least one alcohol or the liquid containing a polysaccharide can be brought about by immersing the balloon in the liquid, similar to the coating with the active substance as described hereinbefore. It is useful to also perform these wetting steps at the pressure at which also the coating with the active agent is performed, in order to ensure a uniform detachment of the coating in the event of greater expansion of the balloon at the target site in the blood vessel. In this context, a pressure that differs slightly from the pressure at which the coating with active agent is carried out is also regarded as an identical pressure, provided that the balloon diameter is largely the same. As an alternative to wetting the balloon by immersion, other methods may also be adopted as mentioned hereinbefore, for example by spraying. After the individual coating steps, a drying step is advisable. In the event of volatile solvents, drying may be immediate; for other solvents, drying may be aided by rotating the balloon or by producing an air stream.

In principle, certain coating steps can also be repeated. It is conceivable, for instance, to bring the balloon into contact with an active agent solution several times with a view to increasing the active agent loading. If applicable or necessary, it is also possible in this context to apply different active agents.

In addition to the method proposed by the invention, the invention also relates to a balloon provided with a coating such as can be achieved by the method described. This method is characterized by the fact that when the balloon is substantially expanded beyond the degree of balloon expansion at which the coating has been produced, strong shear forces are exerted that cause the coating to spall off and be released and transferred from the balloon surface onto the inner wall of the blood vessel/surrounding tissue. Furthermore, the invention relates to a balloon catheter comprising such a balloon. Depending on their dimensions, balloon catheters can be used in a wide variety of areas of the blood vessel system, that is to say in particular in the coronary, intracranial and peripheral areas.

The balloon catheter provided by the invention typically comprises lumens, preferably at least two lumens, with one lumen serving for the supply of fluid and pressurization and being connected to the interior of the balloon, while the other lumen serves to accommodate a guidewire that is initially pushed forward to the target site, with a view to subsequently moving the balloon catheter to the target site via the guidewire. In this context, essentially two different systems are known from prior art, namely over-the-wire (OTW) and rapid exchange (Rx) balloon catheters. The balloon catheter according to the invention can be either an OTW or an Rx balloon catheter. While in an OTW catheter the lumen for the guidewire extends from proximal to distal along the entire length of the catheter, an Rx catheter is designed to have a separate guidewire access port (Rx port) where the guidewire exits the catheter significantly distal to the proximal end of the catheter. Accordingly, in the case of an OTW balloon catheter, the lumens for fluid delivery and guidewire run parallel or concentric to each other from the proximal end of the catheter up to the balloon, whereas in the case of an Rx catheter, this is only the case between the Rx port and the balloon. On the other hand, the section between the Rx port and the proximal end has only one lumen for fluid delivery. Typically, the lumens extend parallel to each other in the areas where the catheter comprises two lumens. A concentric configuration is also possible, in which the narrower inner lumen for the guidewire passes through the wider outer lumen for the fluid supply.

At the proximal end of the balloon catheter, a so-called catheter hub is usually provided, i.e. a connector for the device serving for fluid supply and pressurization. The connector, for example, can be a conventional luer or luer-lock connection. Proximal is understood to mean toward the outside of the body, i.e., toward the attending physician, while distal shall be understood to denote the opposite direction, i.e., toward the blood vessel being treated. The balloon catheter is usually inserted into the human body in the groin area via the femoral artery.

Over the length of the balloon catheter radiopaque markers may be arranged at various positions, said markers serving the purpose of making the catheter visible on radiographs. In particular, said markers may be manufactured of platinum or a platinum alloy.

### EXAMPLE

A balloon made of an elastic polyurethane is pressurized from the inside with a pressure of 3 bar. At this pressure, the balloon has a diameter of 4.5 mm. In this state, the balloon is immersed into a solution consisting of paclitaxel in methylene chloride and then slowly withdrawn. The concentration of paclitaxel amounts to 200 mg/ml. The coating process takes place at room temperature. Following this, the pressure is released, after which the balloon folds up tightly.

The balloon forms part of a balloon catheter that is inserted into the human body and advanced through the blood vessel system to the target site, where pressurization takes place at 6 bar. At this pressure, the diameter amounts to 6 mm, which means the diameter and surface area have increased by 33% compared to the initial coating step. This causes the development of strong shear forces resulting in the active agent layer to spall off and be transferred to the inner wall of the vessel.

1. Method for coating the surface of the balloon of a balloon catheter with an active substance, wherein the balloon being made of an elastic material and being expandable by pressurization with a fluid, with the balloon being designed for expansion at a target site, wherein the coating of the surface of the balloon being applied at a pressure which is lower than the pressure used to expand the balloon at the target location.

2. Method according to claim 1, wherein the pressure at which coating takes place is at least 20%, preferably at least 30% lower than the pressure that is exerted when the balloon is dilatated at the target site.

3. Method according to claim 2, wherein the pressure at which coating takes place amounts to a maximum of 50% of the pressure that is exerted to expand the balloon at the target site.

4. Method according to claim 1, wherein the elastic material comprises a polyurethane, a polyolefin copolymer, a polyethylene or a silicone.

5. Method according to claim 1, wherein the elastic material comprises a thermoplastic elastomer, in particular a polyether block amide.

6. Method according to claim 1, wherein, starting from the rated pressure at which the balloon reaches its nominal diameter, the diameter increase of the balloon when doubling the pressure is at least 5%.

7. Method according to claim 1, wherein the active agent used is selected from the following group: Tretinoin, orphan receptor agonists, elafin derivatives, corticosteroids, steroid hormones, paclitaxel, rapamycin, tacrolimus, hydrophobic proteins, heparin and/or hormone-like or cell proliferation-modifying substances.

8. Method according to claim 1, wherein at least the part of the balloon surface coated with the active substance is wetted with a liquid containing water and/or at least one alcohol.

9. Method according to claim 1, wherein at least the part of the surface of the balloon coated with the active substance is provided with a coat of polysaccharide before or after the active agent coating is applied.

10. Method according to claim 9, wherein the mean molar mass of the polysaccharide amounts to between 10,000 and 100,000,000 Da.

11. Method according to claim 10, wherein the mean molar mass of the polysaccharide amounts to between 20,000 and 80,000 Da.

12. Method according to claim 9, wherein the polysaccharide is a dextran.

13. Balloon of a balloon catheter, the surface of which is provided at least partially with a coating comprising an active agent obtainable through a method in accordance with claim 1.

14. Balloon catheter comprising a balloon in accordance with claim 13.

15. Method according to claim 6, wherein, starting from the rated pressure at which the balloon reaches its nominal diameter, the diameter increase of the balloon when doubling the pressure is at least 10%.

16. Method according to claim 7, wherein, starting from the rated pressure at which the balloon reaches its nominal diameter, the diameter increase of the balloon when doubling the pressure is at least 20%.

17. Method according to claim 8, wherein, starting from the rated pressure at which the balloon reaches its nominal diameter, the diameter increase of the balloon when doubling the pressure is at least 30%.
